(19) United States
(12) Patent Application Publication
(10) Pub. No.: US 2023/0414377 A1
(43) Pub. Date: Dec. 28, 2023

(54) BONE CEMENT PRESSURISER

(71) Applicant: University of Newcastle upon Tyne, Newcastle upon Tyne, Tyne and Wear (GB)

(72) Inventors: Alexander Thomas Boote, Newcastle upon Tyne, Tyne and Wear (GB); Philip James Hyde, Newcastle upon Tyne, Tyne and Wear (GB)

(73) Assignee: University of Newcastle upon Tyne, Newcastle upon Tyne, Tyne and Wear (GB)

(21) Appl. No.: 18/265,093

(22) PCT Filed: Nov. 24, 2021
(86) PCT No.: PCT/GB2021/053050

§ 371 Date: Jun. 2, 2023

(30) Foreign Application Priority Data
Dec. 3, 2020 (GB) 2019062.5

(51) Int. Cl. A61F 2/46; A61B 17/88

(52) U.S. Cl. CPC A61F 2/4601; A61B 17/8808; A61B 17/8811

(57) ABSTRACT

A bone cement pressuriser (50) comprising a pressuriser head. The pressuriser head has a first side (51) coupled to or configured to couple to an end of a pressuriser handle and a second side (52) configured to transfer pressure to bone cement in a bone cavity. The second side of the pressuriser head is defined by a first, convex curved surface (53) and a second surface (54) forming a ring surrounding the first surface and extending radially beyond the first surface. A method of pressurising bone cement using the bone cement pressuriser is also disclosed. The first surface is configured to pressurise bone cement within a bone cavity and the second surface is configured to both pressurise bone cement within the bone cavity and to seal about the periphery of the bone cavity.

### TECHNICAL FIELD

The present invention relates to a bone cement pressuriser, and particularly to a bone cement pressuriser with a novel shape of pressuriser head. A further aspect of the invention relates to a method of pressurising bone cement using the novel bone pressuriser.

### BACKGROUND

As illustrated in image (a) of FIG. 1, on the right hand side, the natural hip joint consists of an articulating ball and socket joint comprising a femoral head 1 and an acetabulum 2. The acetabulum 2 is a concave surface formed within the pelvis 3, within and against which the femoral head 1 articulates. The femoral head 1 is connected to the femur 4 via the femoral neck 5.

A total hip arthroplasty involves removal of a diseased (usually arthritic) hip joint to make way for replacement femoral and acetabular components. The left hand side of image (a) of FIG. 1 illustrates a replacement hip joint 6 comprising a femoral implant 7 (the neck and head of which are partially exposed) and an implanted acetabular cup 8. The femoral implant 7 may be provided as severable stem and head components. The femoral stem is typically made from metal alloys of titanium and cobalt chromium. The femoral head may be made either of a cobalt chromium alloy or a ceramic material. The acetabular cup 8 is predominately made from a plastic (for instance, polyethylene) or a metal alloy with a plastic liner.

Images (b), (c) and (d) of FIG. 1 schematically illustrate the process of performing total hip arthroplasty. Image (b) is a diseased hip. Image (c) illustrates hip removal: typically comprising removing the femoral head and neck, reaming the femoral medullary canal to accept the femoral implant stem, and reaming the acetabulum to accept the acetabular cup. Image (d) illustrates implantation of the femoral implant and 7 and the acetabular cup 8. The present invention concerns the implantation of an acetabular cup and so the femoral implant will not be further described.

In approximately 30% of all hip replacements performed in England, Wales, Northern Ireland, the Isle of Man and Guernsey in 2019 the acetabular cup is anchored into a prepared acetabulum using bone cement (National Joint Registry Report 2019). Cement is analogous to tiling grout as opposed to an adhesive and is used to anchor an acetabular cup in place. To achieve this anchorage, it is conventional to first pressurise the cement into the pelvic bone to get a good mechanical interlock. Preparation of the acetabulum typically comprises reaming the acetabulum using a hemispherical reamer to remove bone thereby forming a larger socket to receive the acetabular cup. Reaming also exposes porous trabeculae bone. Bone cement starts as a liquid acrylic plastic which solidifies over the operation period. At the point when the bone cement has achieved the consistency of dough it is forced into exposed porous bone within the reamed acetabulum and pressurised using a cement pressuriser (as will be described below in connection with FIG. 2). Applying pressure to the bone cement promotes interdigitation between the bone cement and bone pores in order to anchor the bone cement (and hence the acetabular cup) into prepared acetabulum. After the pressurisation stage the acetabular cup is implanted by pressing it into the bone cement.

During acetabular cup insertion a large amount of bone cement is displaced. This excavation of cement while still viscous results in residual stresses in the final solidified cement which reduces its strength. Weakened cement-bone interface may be observed during post-operative radiograph inspection of cemented acetabular components as radiolucent lines revealing a weakened connection between cement mantle and bone around the rim of the acetabular cup. This weakened connection is an identified failure mode of cemented acetabular cups: failure occurs at the interface between bone and bone cement rather than failure at the interface between the bone cement and the acetabular cup. Accordingly, observed radiolucent lines are an indicator of an increased risk of early cup loosening.

It is an aim of certain examples of the present invention to solve, mitigate or obviate, at least partly, at least one of the problems and/or disadvantages associated with the prior art. Certain examples aim to provide a stronger connection between cement mantle and bone for an implanted acetabular cup to reduce the risk of implant failure.

### BRIEF SUMMARY OF THE INVENTION

The scope of the present invention is defined by the appended claims.

Aspects of the invention provide a bone cement pressuriser, and a method of pressurising bone cement as claimed in the appended claims. Particular examples of the present invention aim to reduce the failure rate of cemented acetabular cups by modifying the cement pressurisation stage. Particularly, a bone cement pressuriser with an improved shape is provided which aims to remove excess cement at the pressurisation stage as opposed to the implant insertion stage, resulting in a stronger cement/bone bond.

The present inventors have quantified uneven pressure distribution during cement pressurisation using conventional bone cement pressurisers and during acetabular cup insertion, as well as the difference in tensile strength between cement that was deformed late in the curing process and cement that was allowed to rest during the curing process. Based on this data, the present inventors have developed a new shape for a bone cement pressuriser head. The new shape of pressuriser head decreases excess bone cement that has to be removed when pressing the acetabular cup into the bone cement. Consequently, bone cement deformation occurs at an earlier surgical stage such that the deformation weakens bone cement to a lesser extent. This improves the final mechanical interlock between bone, cement and acetabular cup.

While the development of the present invention derives from data observed for implanted acetabular cups, the present invention is not limited only to hip replacement surgery. In particular, the same considerations apply also to other orthopaedic arthroplasty procedures, for example anatomical total shoulder replacement, where there is a similar need to pressurise bone cement to achieve interdigitation before inserting an implant into the bone cement.

According to a first aspect of the present invention, there is provided a bone cement pressuriser comprising: a pressuriser head having a first side coupled to or configured to couple to an end of a pressuriser handle and a second side configured to transfer pressure to bone cement in a bone cavity; wherein the second side of the pressuriser head is defined by a first, convex curved surface and a second surface forming a ring surrounding the first surface and extending radially beyond the first surface. The first surface may be configured to pressurise bone cement within a bone cavity and the second surface may be configured to both pressurise bone cement within the bone cavity and to seal about the periphery of the bone cavity.

The pressuriser head may comprise a filled volume between the first and second sides.

The second surface may flare outwards from the first surface.

The second surface may extend directly from the first surface to define a continuous surface. The first and second surfaces may be uninterrupted

The pressuriser head may be shaped to pressurise bone cement in an acetabulum.

The first surface may be shaped to excavate bone cement within the acetabulum under the application of pressure.

The second surface may be shaped to seal the periphery of the acetabulum to minimise the egress of bone cement.

The second surface may also be a convex curved surface, the first surface being more tightly curved than the second surface.

The first surface may be domed or defined by a spheroidal cap.

The first and second surfaces may form a double-domed surface.

The first or second surface may be rotationally symmetrical.

The first surface may be defined by a spheroidal cap having a first focus defining a first polar axis; and the second surface may be defined by a spheroidal segment having a second focus located on the first polar axis. The first polar axis may pass through the first focus and through the first surface. Particularly, it may pass through the centre of the first surface, however in some examples the first polar axis may pass through some other point within the first surface. In some examples the arrangement of first and second foci may be such that the spheroidal segment is rotationally symmetrical about the first polar axis. In certain examples the first and second surfaces (for instance, but not necessarily, a spheroidal cap and a spheroidal segment) may be aligned such that the whole pressuriser head is rotationally symmetrical. However, the present invention is not limited to this. The first surface may be offset from a centre of the second surface.

The first and second centres may be spaced apart along the first polar axis. However, it may be that at least one of the first and second centres is not aligned with the first polar axis.

The second centre may be further from the second side of the pressuriser head than the first centre.

The gap between the first and second centres may be greater than or equal to about 5 mm.

The spheroidal cap may be a spherical cap having a first radius between about 15 mm and about 40 mm.

The first radius may be approximately 26 mm.

The spheroidal segment may be a spherical segment having a second radius greater than or equal to about 20 mm.

The maximum width of the second surface along an axis normal to the first polar axis may be between about 37 mm and about 150 mm.

The pressuriser head may further comprise a flange surrounding the second surface or an asymmetric protrusion extending from the second surface.

The bone cement pressuriser may further comprise a pressuriser handle configured to couple to the first side of the pressuriser head.

The pressuriser handle may extend at least partially along the first polar axis. However, in other examples it need not be aligned. More generally, the first surface, the second surface and the pressuriser head may have any arbitrary spatial arrangement so long as the functional requirements of each part are fulfilled.

The pressuriser head may be elastically deformable. The pressuriser head may be formed from a viscoelastic material with a Shore A hardness between about 0 and 90.

The pressuriser head may be formed from silicone.

According to a second aspect of the present invention, there is provided a method of pressurising bone cement, the method comprising: preparing a bone cavity for receiving bone cement; inserting bone cement into the bone cavity; and pressurising the bone cement in the bone cavity using a bone cement pressuriser according to any one of the preceding claims by bearing the second side of the pressuriser head against exposed bone cement in the bone cavity and applying pressure to the pressuriser head through a coupled pressuriser handle. The first surface may be configured to pressurise bone cement within a bone cavity and the second surface may be configured to both pressurise bone cement within the bone cavity and to seal about the periphery of the bone cavity.

### BRIEF DESCRIPTION OF THE DRAWINGS

Examples of the invention are further described hereinafter with reference to the accompanying drawings, in which:

FIG. 1 illustrates in image (a) a natural hip joint (right hand side) and a replacement hip joint (left hand side), and in images (b), (c) and (d) the process of replacing a hip joint;

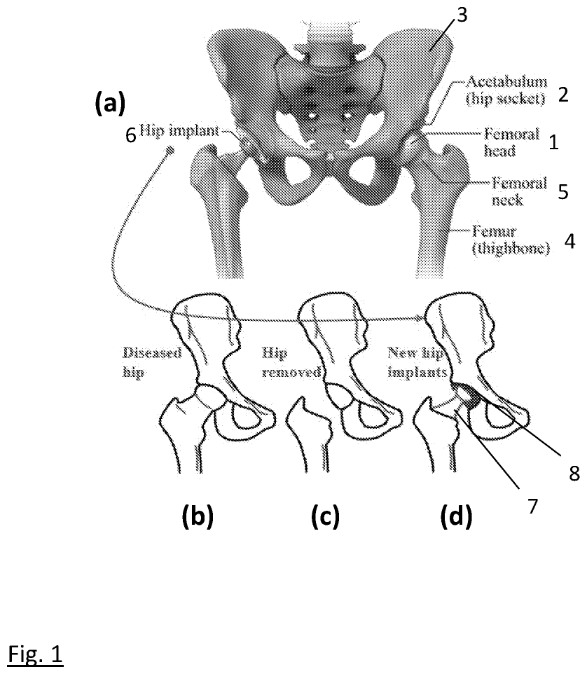

FIG. 2 illustrates in cross section views a conventional process of surgically implanting a replacement acetabular cup;

FIG. 3 is a graph illustrating temperature and pressure over time for bone cement during surgical implantation of an acetabular cup when the bone cement is pressurised using a conventional bone cement pressuriser;

FIG. 4 is a box plot showing the difference in ultimate tensile strength due to deforming cement during curing;

FIG. 5 is a side by side comparison of a conventional bone cement pressuriser and a bone cement pressuriser according to an example of the present invention;

FIG. 6 illustrates a prototype silicone bone cement pressuriser according to an example of the present invention;

FIG. 7 is an annotated cross section of a bone cement pressuriser according to an example of the present invention;

FIGS. 8a and 8b illustrate a further example of a bone cement pressuriser according to the present invention; and

FIGS. 9a and 9b illustrate a yet further example of a bone cement pressuriser according to the present invention.

### DETAILED DESCRIPTION

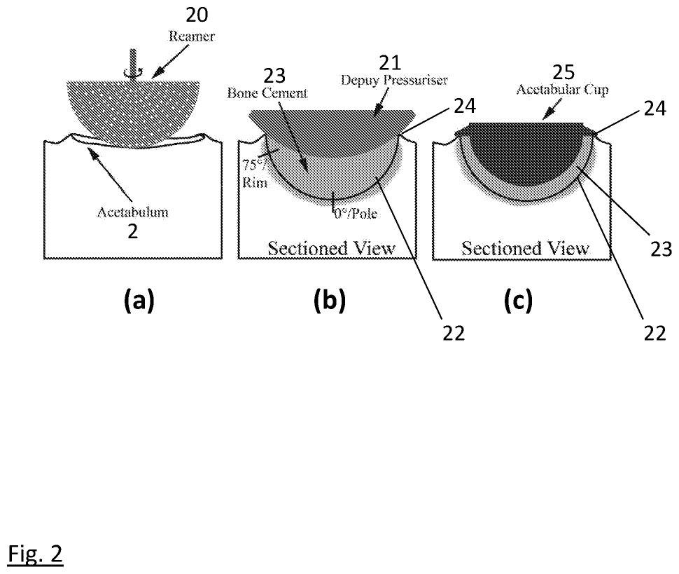

Referring to FIG. 2, a conventional process of surgically implanting an acetabular cup will now be described in greater detail.

Initially, as illustrated in image (a) a rotating hemispherical reamer 20 is used to remove diseased and damaged bone in the acetabulum 2 and to expose the porous trabeculae bone in the acetabulum 2. Trabeculae bone provides an ideal surface for interdigitation with bone cement. After bone reaming the liquid acrylic plastic bone cement is mixed, and when at the correct dough-like consistency the cement is placed into the reamed acetabular cavity. Most bone cements used for acetabular cup fixation are based on PMMA (polymethyl-methacrylate). The cement comes as two components: a powder, which is primarily ground PMMA and a liquid which is primarily MMA monomer. When the two components are mixed a polymerisation reaction starts which continues until full cure and rigidification.

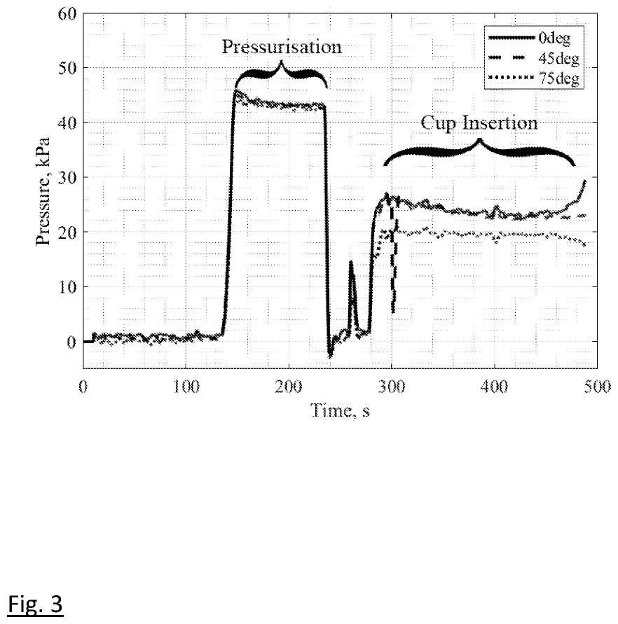

As illustrated in image (b) a bone cement pressuriser 21 is used to seal the reamed acetabular hemisphere 22 and through applying force, pressurise the bone cement 23. Particularly, a DePuy Synthes Smartseal pressuriser is shown. This is intended to force the cement 23 into the exposed trabeculae bone about the acetabular hemisphere 22 and achieve fixation through interdigitation. The pressuriser comprises pressuriser head having a convex curved surface, which may comprise a spherical cap, sized in order to seal about the periphery 24 of the acetabular hemisphere 22. Sealing the periphery 24 serves to prevent or minimise the egress of bone cement 23 from the acetabular hemisphere 22 during pressurisation. Although not illustrated, the reverse of the pressuriser head is coupled to a pressuriser handle to permit a surgeon to transfer force to the pressuriser head. Image (b) also indicates a polar axis for the reamed acetabular hemisphere 22 (0°) and also a position with a 75° polar angle (at the centre of the hemisphere, relative to the polar axis) which is close to the periphery 24. These polar angles are referenced in the graph of FIG. 3, discussed below.

As illustrated in image (c) the pressuriser 21 is then removed, and the prosthetic acetabular cup 25 is inserted. Force is applied to the acetabular cup 25 until enough excess bone cement is displaced for the cup to be positioned correctly. During the insertion of the acetabular cup 25 the applied force serves to displace the bone cement 23 out of the acetabular hemisphere 22.

In 2018, the top five most common cemented acetabular cups were manufactured by Stryker and DePuy Synthes. Both Stryker and DePuy Synthes include within their surgical instrumentation kits for the implantation of the acetabular cup an acetabular bone cement pressuriser handle (either straight or curved and typically metallic), which is reusable and pressuriser heads (also referred to as acetabular cement seals) in a range of varying sizes corresponding to different size acetabular cups. The pressuriser heads are single-use, disposable items, typically formed from silicone.

It will be appreciated from inspection of images (b) and (c) of FIG. 2 that during insertion of the acetabular cup 25 into the bone cement 23 a significant proportion of the bone cement is displaced and egresses the acetabular hemisphere 22 about the periphery 24. The flow of the cement out of the acetabular hemisphere 22 during acetabular cup insertion results in a pressure differential between a polar position and a rim position, particularly a lower cement pressure at the rim, this pressure gradient being implicit for any flowing fluid. This displacement of bone cement takes place during a later part of the surgical procedure at which time the bone cement has solidified further than during the pressurisation stage. The present inventors have identified that bone cement is weakened when it is deformed during surgery before it has fully hardened.

Referring now also to the graph of FIG. 3 (and with continued reference to the polar angles illustrated in image (b) of FIG. 2, and an intervening 45° polar angle) this shows the cement pressure profile at the surface at the acetabular hemisphere 22 during a surgical procedure to implant an acetabular cup using a conventional bone cement pressuriser as illustrated in image (b) of FIG. 2. 0° is the bottom, or pole, of the acetabular cup and 75° is proximal to the rim. The graph of FIG. 3 is generated based on data obtained by the present inventors from in vitro implantation of cemented acetabular cups into a model acetabulum. A known form of bone cement pressuriser, such as the DePuy Synthes Smartseal pressuriser illustrated in FIG. 2.

The X axis is time in seconds from when the bone cement is mixed and the Y axis is measured pressure at the surface of the model acetabulum. The solid line is the pressure at the pole, the dashed line is the pressure at a polar angle of 45° and the dotted line is the pressure at a polar angle of 75°. The pressurisation stage and the cup insertion stage are indicated.

It can be seen that during the pressurisation stage the bone cement is evenly pressurised across all three angular positions (to approximately 40 kPa to 45 kPa). This result is intuitive given that the bone cement pressuriser seals the acetabulum to prevent cement egress (after any excessive quantity of bone cement applied by the surgeon has been displaced) and so the bone cement is a closed volume.

However, during cup insertion it can be seen that the pressure at the 75° position—that is, at the rim of the acetabular hemisphere 22—is appreciably lower than the pressure closer to the pole 0°, though all are lower than during the pressurisation stage. This lower pressure at the rim results from the flow of cement out of the reamed cavity, as described above.

From the cement pressure data it is evident that existing bone cement pressurisers in current usage result in an excess of cement which has to be removed when the acetabular cup implant is inserted. This removal of cement causes residual stresses in the cement and results in weakness at the rim area of the cement mantle.

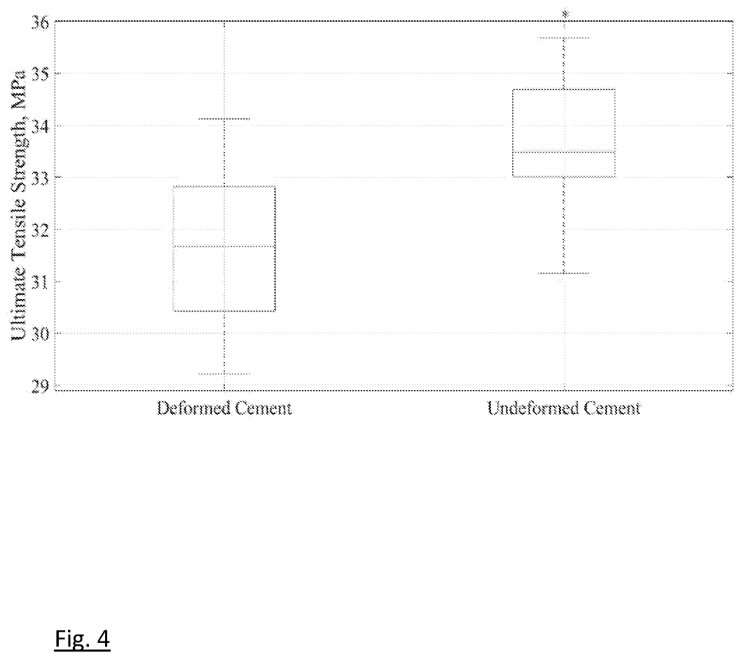

The present inventors have identified that bone cement is weakened when it is deformed during cement curing, particular when it is deformed during cup insertion. FIG. 4 is a box plot displaying the ultimate tensile strength of bone cement which is deformed during curing (left hand side) and bone cement which is un-deformed during curing (right hand side). The ultimate tensile strength is indicative of the fatigue strength of bone cement which is the most likely failure mode of the cement mantle during time in vivo. It will be understood that bone cement deformation is greatest proximal to the periphery of the acetabular hemisphere as bone cement in this region undergoes the greatest flow as it displaced during cup insertion. The present inventors have identified that bone cement is weaker if subject to deformation as it approaches the cure point (that is, bone cement is displaced during cup insertion) compared with if the bone cement is static as it approaches the cure point.

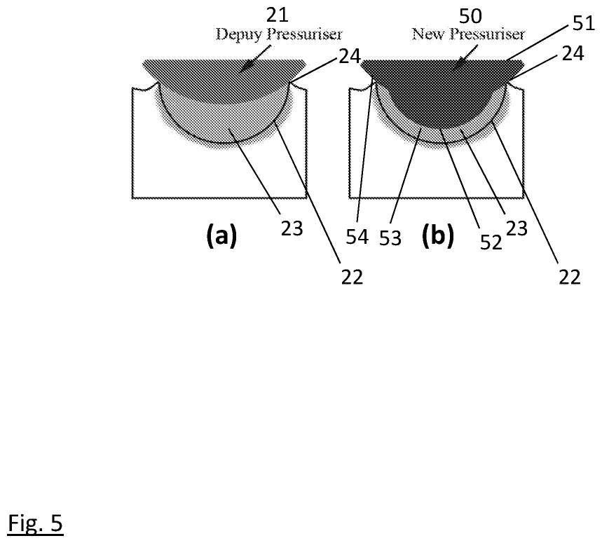

This cement weakening is considered to be a result of the increase of an equilibrium modulus. The larger the equilibrium modulus, the larger is the resulting stress for an equivalent applied strain. It is known that for bone cement the equilibrium modulus increases as the extent of polymerisation increases. Accordingly, the earlier in the procedure the cement is deformed, the lower the resulting residual stress will be. The present inventors have therefore identified that so far as possible, necessary bone cement deformation (so that the bone cement conforms to the shape of the acetabular implant) should occur as early as possible during the surgical procedure. This insight has informed the development of a new form of bone cement pressuriser head described below beginning with FIG. 5. The new bone cement pressuriser head is intended to shape the bone cement appropriately for receiving the acetabular cup while the bone cement is more pliable. By appropriately, it is meant that the shape of the exposed bone cement is closer to the shape of the acetabular cup than is presently the case using conventional bone cement pressurisers. Consequently, late stage bone cement deformation is reduced. It will be appreciated that a certain degree of bone cement deformation during acetabular cup insertion is unavoidable if a strong bond between bone cement and acetabular cup is to be achieved.

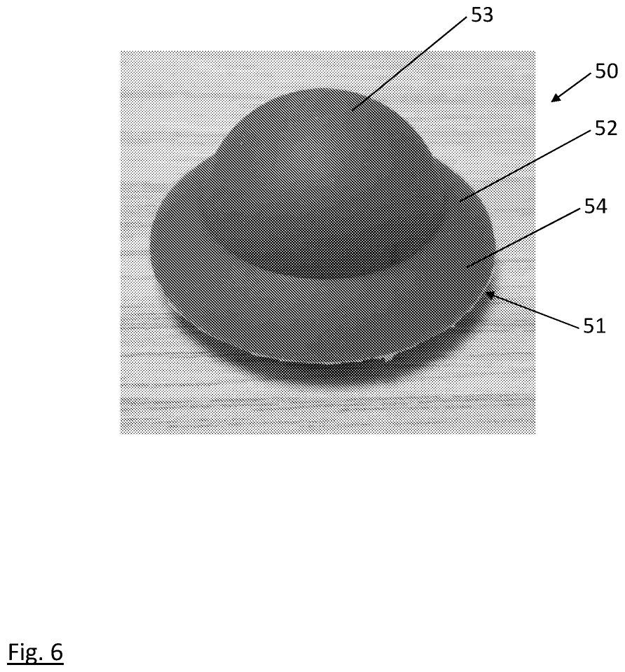

Referring now to FIG. 5, this illustrates in cross section a conventional bone cement pressuriser 21 (left hand, image (a)) and a bone cement pressuriser 50 according to an example of the present invention (right hand, image (b)). Particularly, images (a) and (b) show the respect bone cement pressuriser heads. In use a pressuriser handle is affixed to the side of the pressuriser head facing away from the bone in order to allow a surgeon to exert pressure, however as the handle and how it affixes to the pressuriser head may be entirely conventional it is not further described or illustrated. Image (a) is the same as image (b) of FIG. 2 and is repeated for side by side comparison with the bone cement pressuriser 50 according to an example of the present invention. FIG. 6 is a perspective image of a prototype bone cement pressuriser 50 according to an example of the present invention formed from silicone.

Image (a) in FIG. 5 uses as example of a conventional bone cement pressuriser 21 the commonly used Smartseal pressuriser (DePuy Synthes, UK) which is manufactured using silicone as for the example bone cement pressuriser 50 of FIG. 6. The DePuy Synthes Smartseal pressuriser is the same as is illustrated in FIG. 2. As previously described, the Smartseal pressuriser comprises a convex curved surface facing the acetabulum. The surface is continuously curved, and comprises a spherical cap with a fixed radius across the whole area facing the acetabulum. The primary function of the Smartseal (as indeed is implied by its name) is to seal about the periphery 24 of the reamed acetabular hemisphere 22 in order to minimise the egress of bone cement 23 while pressurising the bone cement.

In stark contrast, following the insights described above from the present inventors, the bone cement pressuriser 50 according to an example of the present invention has two distinct functions (in addition to the transfer of pressure to the bone cement): firstly to seal the periphery of the acetabulum to minimise the egress of bone cement during pressurisation (as for the conventional pressuriser 21), and secondly to shape the bone cement 23 during pressurisation to conform more closely to its final required shape when an acetabular cup is inserted. This second function is the basis of the present invention. Accordingly, the bone cement pressuriser head 50 serves to improve the pressurisation process by reducing the amount of excess bone cement that has to be removed once the acetabular cup is inserted. In consequence, residual stresses in the solidified bone cement are minimised and pressure proximal to the rim of the cup is increased during cup insertion for an cemented acetabular cup. This can improve the resulting longevity of the bone cement mantle and the mechanical interlock with the bone.

Bone cement pressuriser head 50 comprises a first side 51 (uppermost in image (b) of FIG. 5 and underneath (and hidden from view) in FIG. 6 which is either permanently coupled to a pressuriser handle (not shown) or configured to be releasably coupled a pressuriser handle. The coupling mechanism is not germane to the present invention, but for instance the pressuriser head first side 51 may have a socket that matches the tip of the handle or the handle may have a small lip at the end and a corresponding negative shape on the head. Other options will be apparent to the skilled person, for instance a bayonet fit. Other than permitting coupling to the pressuriser handle, the shape of the first side 51 has no bearing on the pressurisation of bone cement, and may simply be flat, as illustrated.

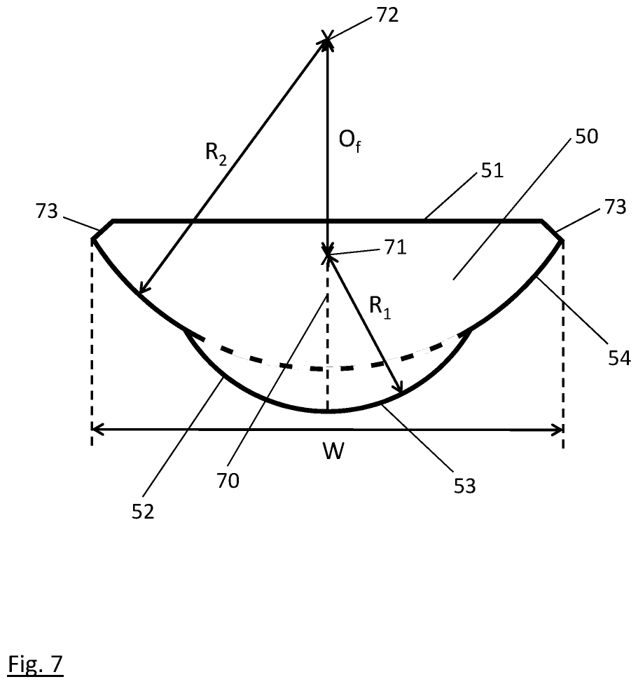

In contrast, the second side 52 facing into the acetabulum is specifically shaped to excavate more cement at the pressurisation stage thereby improving pressure distribution. The second side 52 transfers pressure to bone cement 23 in the bone cavity. The second side 52 of the pressuriser head 50 is defined by a first, convex curved surface 53 and a second surface 54 forming a ring surrounding the first surface 53 and extending radially beyond the first surface 53. The convex first surface 53 is selected to have a shape similar or identical to a curved outer surface of an acetabular cup to be subsequently inserted into the shaped bone cement. In some examples the first surface 53 serves to excavate bone cement to form a depression slightly larger than the outer shape of the acetabular cup. Excavating a depression slightly larger than the outer shape of the acetabular cup ensures that during cup insertion a cup surface close to the pole contacts the bone cement first such that any cement flow is towards the rim. In contrast, were the excavated depression to be slightly smaller than the outer shape of the acetabular cup, the rim of the cup would contact the bone cement before the pole, risking a cavity being trapped between the cup and the bone cement. The offset between the centre of the first curved surface and the centre of the second curved surface (described below in connection with FIG. 7) means that the excavated depression may be shallower than the final inserted cup position, which permits a small amount of bone cement 23 to be displaced during cup insertion (that is, a small amount of excess cement is conserved and then removed during cup insertion), permitting a strong bond to be formed between the bone cement and the cup.

The second side 52 comprises the first surface 53 and the second surface 54. The first surface 53 may be a spheroidal cap. The second surface 54 may be a spheroidal segment. Alternatively the second surface 54 may be a conical section or, more generally, a flange of any shape such that it extends radially further than the first surface 53. The second surface 54 is continuous from the first surface 53. That is, there is no intervening portion. The second side 52 may be completely defined by the first and second surfaces 53, 54: there may be no further component surfaces. The first and second surfaces may be unbroken—that is they may not be interrupted for instance by an aperture.

Where both surface 53, 54 are curved, the curvature may be different and the radius of curvature may change at the line where the surfaces intersect. Where both surfaces are curved, they may both be convex, that is both curving in the same direction (albeit with different radii and/or different focuses). The first and second convex surfaces 53, 54 may intersect at a convex vertex. Where the first surface 53 is a spheroidal cap and the second surface 54 is a spheroidal segment, the first and second surfaces 53, 54 meet at a convex vertex defining a circle (assuming that the spheroidal segment has a focus located on a polar axis defined by the spheroidal cap).

As will be made clear from the following description, the purpose of the first surface 53 is to pressurise cement present in the acetabular cup. The second surface 54 may have the twin purposes of pressurising cement present in the acetabular cup while also sealing about the rim of the acetabular cup in order to maintain cement pressure.

The pressuriser head 50 may be solid or filled. The pressuriser head 50 may comprise a filled volume between the first and second sides 51, 52. That is most, all or substantially all of the pressuriser head 50 may be filled within a volume defined by the second side 52 and a plane closing off the second side.

This excavation of cement to receive the acetabular cup at the pressurisation stage rather than during cup insertion reduces the amount of cement that needs to be displaced for correct acetabular cup insertion. This reduces residual stresses in the cement caused by cement movement during cup insertion when the cement is still setting but less pliable (relative to the pressurisation stage, as more time has elapsed since cement mixing) and hence improves strength and fatigue strength. The use of the new design of pressuriser head 50 allows for the removal of excess cement during pressurisation before acetabular cup insertion. Advantageously, this means the majority of necessary cement flow occurs earlier in the solidifying of the cement. Experimentation has shown that this improves strength and eliminates the creation of weakness within the cement mantle when compared to the existing techniques.

While the first surface 53 is shaped to excavate bone cement 23, the second surface 54 is primarily intended to perform the sealing function about the periphery 24 of the reamed acetabulum. As is particularly evident in FIG. 6, the second surface 54 flares outwards from the first surface 53. To seal the acetabulum the second surface 54 extends out radially further than the first surface 53. The second surface 54 may be also be a convex curved surface. If so, it may be less tightly curved than the first surface 53 (as illustrated), thus permitting the second surface to extend radially further in a shorter distance along a polar axis. However, it need not be curved. The second surface 54 may be a conical section: that is, flat in cross section. It may extend from the first surface 53 at any angle relative to the polar axis defined by the first surface 53, including extending normally to the polar axis (that is, horizontally in the cross section of FIG. 5). So long as it performs a sealing function, the second surface 54 may be essentially any shape.

As is particularly clear in FIG. 6, there may be a distinct interface between the first and second surfaces 53, 54. This may take the form of a discontinuity in the curvature, or a crease. In some examples the interface between the first and second surfaces may be somewhat smoothed or blended, rather than being a sharp discontinuity.

Again, as is particularly clear in FIG. 6, the first surface 53 may be domed. In combination the first and second surfaces 53, 54 may form a double-domed surface. Either or both of the first and second surfaces 53, 54 may be rotationally symmetrical. Alternatively, either surface 53, 54 may have a curvature or inclination that varies about the circumference of the second side 52 of the pressuriser head 50.

It is not necessary that either surface 53, 54 has a precise geometric shape. The first surface 53 is convex and as described above intended to at least approximate the shape of an acetabular cup to be inserted. The second surface 54 may be more freely selected still. However, in certain examples either or both surfaces 53, 54 may be defined by spheroids. As used herein, the term spheroidal is intended to encompass all possible spheroidal shapes, including but not limited to spheres, ellipsoids and paraboloids. Clearly the first and second surfaces 53, 54 may also be defined by alternative geometric shapes.

For instance, the first surface 53 may be defined by a spheroidal cap having a first focus defining a first polar axis. Similarly, the second surface 54 may be defined by a spheroidal segment having a second focus located on the first polar axis. It is not necessary that both surfaces 53, 54 are defined by spheroids: for instance, the first surface 53 may be while the second surface 54 may be defined by a conical section.

Referring now also to FIG. 7, this illustrates a specific shape of pressuriser head 50 in which the first surface 53 comprises a spherical cap defined by a first radius R₁ and the second surface 54 comprises a spherical segment defined by a second radius R₂. The first surface 53 defines a polar axis 70 upon which is located the first centre 71 of the first surface 53. A second centre 72 for the second surface 54 may also be located on the polar axis 70. In an alternative example whereby the first and second surfaces 53, 54 are defined by a spheroidal cap and a spheroidal segment, it may be that the focus for the spheroidal cap define a polar axis and the focus of the spheroidal segment are located on the same polar axis.

FIG. 7 also illustrates the first side 51 of the pressuriser head 50. The first and second sides 51,52 are joined via a chamfer 73, though alternatives are possible, including that they directly connect, or are joined by a surface parallel to the polar axis 70, a further curved, or a step. The interconnection between the first and second sides 51,52 is not critical to the functioning of the pressuriser head 50.

The first surface 53 may comprise a spherical cap with a radius R₁ between about 15 mm and about 40 mm. For instance, R₁ may be about 31 mm. In order for surface 53 to correspond to the shape of a hemispherical acetabular cup to be inserted, radius R₁ is constrained such that it is greater than or equal to the smallest possible radius of cup to be implanted and less than or equal to the largest possible radius of cup to be inserted. A typical acetabular cup may have a radius of approximately 26 mm.

The second surface 54 may comprise a spherical segment with a radius R₂. In order to seal the acetabulum during cement pressurisation, R₂ is greater than or equal to the smallest possible radius of a reamed acetabulum. For instance, greater than or equal to about 20 mm. There is no constraint to the maximum radius R₂ (it may be infinite—a flat surface). It will be appreciated that the greater the radius R₂, the greater the extent to which the second surface approximates a flat surface extending radially.

As illustrated in FIG. 7, the first and second centres 71, 72 are spaced apart along the first polar axis 70. The second centre 72 is further from the second side 52 of the cement pressuriser 50 than the first centre 71. This is illustrated in FIG. 7 as offset O_{f}. Offset O_{f} is greater than or equal to R₂−R₁, and may have a typical minimum of 5 mm. Offset O_{f} is less than or equal to √(R₂²−R₁²). This limit for O_{f} derives from the maximum offset being one which permits surface 53 to form a complete hemisphere such that at the intersection between the first and second surfaces 53, 54 a right angled triangle is formed from O_{f}, R₁ and R₂. Given that R₂ may be infinite, so too may be O_{f} (that is, there is no maximum offset).

The overall width W of the bone cement pressuriser 50 must be such that the full diameter of the reamed acetabulum is covered to minimise bone cement egress. For the smallest possible reamed acetabulum, this suggests a minimum width of around 37 mm (at least for current surgical procedures). The upper limit is dictated only by what is practically required for a large reamed acetabulum before the overlap on either side becomes unwieldy. For instance, the maximum width W may be about 150 mm.

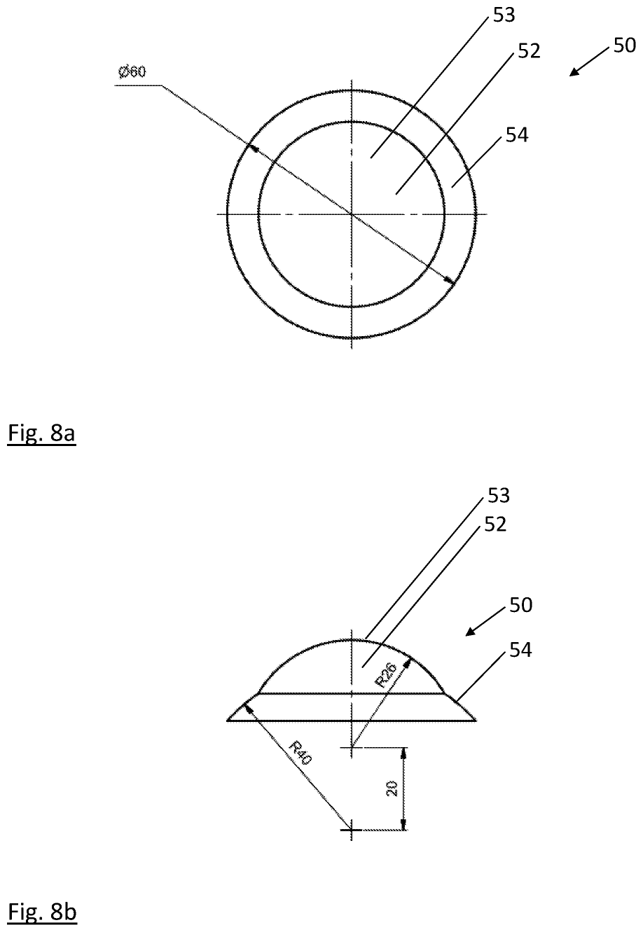

Referring now to FIGS. 8a and 8b, this illustrates a further example of a pressuriser head 50 according to the present invention. FIG. 8a shows a plan view of the second surface 52 of the pressuriser head 50, particularly showing the first curved surface 53 and the second curved surface 54. FIG. 8b is a side view. Dimensions, including radii are given in millimetres. It can be seen that the R₁ is 26 mm and R₂ is 40 mm, but the second surface 54 is cut off at a maximum width of 60 mm. There is a 20 mm offset between the centres of the first and second surfaces 53, 54.

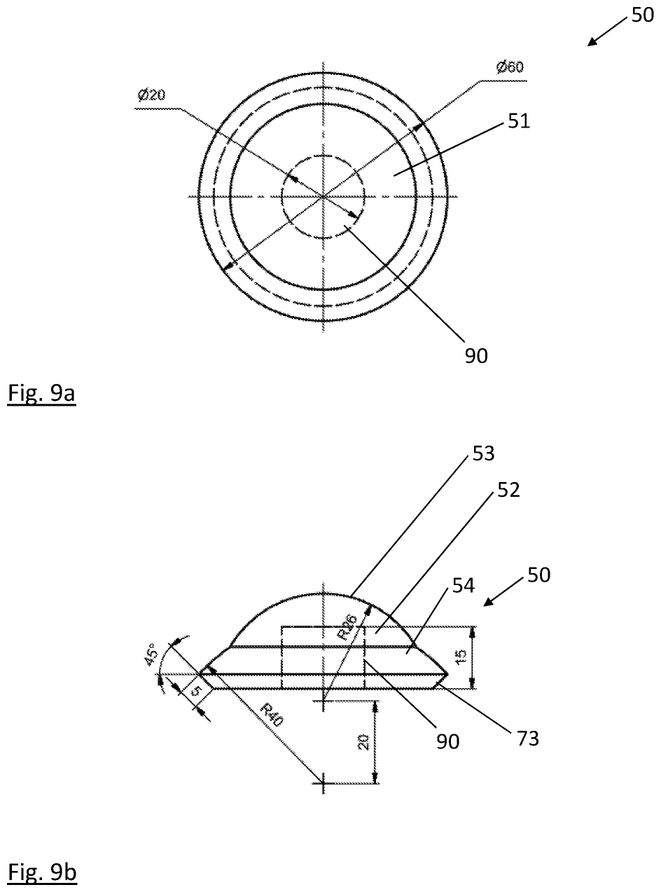

FIGS. 9a and 9b, illustrate a yet further example of a pressuriser head 50 according to the present invention. This differs from the example of FIGS. 8a and 8b by the inclusion of chamfer 73, and also by the explicit illustration a socket 90 for receiving a pressuriser handle. FIG. 9a shows a plan view of the first surface 51 of the pressuriser head 50, particularly showing the socket 90. FIG. 9b is a side view. Dimensions, including radii are given in millimetres.

For the example bone cement pressuriser head 50 illustrated in FIG. 7, a coupled pressuriser handle may extend at least in one part along the polar axis 70.

In some examples, the cement pressuriser head 50 may further comprise a flange surrounding the second surface. As well or instead an asymmetric protrusion or flap may extend from the second surface. The protrusion may assist in sealing the acetabular notch.

As noted above, the bone cement pressuriser head may be formed from silicone. Advantageously, silicone is a resilient material and so will conform to the periphery of the reamed acetabulum even in the event of a degree of unevenness. Preferably the pressuriser head is elastically deformable. This provides the advantage of permitting the second surface 54 to seal about the rim of the acetabular cup (especially where this may have an uneven shape). Additionally, being elastically deformable assists in the distribution of pressure through the cement and movement of cement through the acetabular cup as pressure is applied. While in certain examples the pressuriser head is elastically deformable, and this provides the previously noted advantages, the pressuriser head is substantially a fixed shape. That is, the pressuriser head is not an active component designed to change shape or be mechanically driven to adapt to an acetabular cup, rather its shape is generally fixed apart from the flexibility to conform to the edges of a bone cavity. The pressuriser head is not dependent upon a change in shape or configuration in order to pressurise bone cement. Nor in certain examples is the bone cement pressurisation dependent upon changing the volume of bone cement in a bone cavity (by acting to expel bone cement from the cavity). In certain examples the volume of the pressuriser head is unchanging in use, even where portions of the pressuriser head may flex or deform to accommodate the shape of the bone cavity.

More generally, the pressuriser head may be formed from a viscoelastic material with a Shore A hardness between about 0 and 90. Where silicone is used, it may for example be AS40 addition cure silicone. Other known types of silicone, particularly those known for use in surgical instruments, will be available to the skilled person. In some examples the same type of silicone may be used throughout the pressuriser head. In other examples, different materials may be selected to form different parts of the pressuriser head. As an example, a softer material may be used to form the portion of the pressuriser head 50 defining the second surface 54 in order to better effect a seal about the reamed acetabulum, without compromising the impression into the bone cement formed by the first surface 53.

According to a further example of the present invention, a method of pressurising bone cement is provided. The method comprises preparing a bone cavity for receiving bone cement, for instance by reaming a cavity into an acetabulum. Bone cement is inserted into the bone cavity. The bone cement is then pressurised in the bone cavity using a bone cement pressuriser as described above according to an example of the present invention by bearing the second side of the pressuriser head against exposed bone cement in the bone cavity and applying pressure to the pressuriser head through a coupled pressuriser handle. An acetabular cup may then be inserted into the bone cement.

Throughout this specification, the words “comprise” and “contain” and variations of them mean “including but not limited to”, and they are not intended to (and do not) exclude other components, integers or steps. Throughout this specification, the singular encompasses the plural unless the context otherwise requires. In particular, where the indefinite article is used, the specification is to be understood as contemplating plurality as well as singularity, unless the context requires otherwise. Throughout this specification, the term “about” is used to provide flexibility to a range endpoint by providing that a given value may be “a little above” or “a little below” the endpoint. The degree of flexibility of this term can be dictated by the particular variable and can be determined based on experience and the associated description herein.

Features, integers or characteristics described in conjunction with a particular aspect or example of the invention are to be understood to be applicable to any other aspect or example described herein unless incompatible therewith. All of the features disclosed in this specification, and/or all of the steps of any method or process so disclosed, may be combined in any combination, except combinations where at least some of such features and/or steps are mutually exclusive. The invention is not restricted to the details of any foregoing examples. The invention extends to any novel feature or combination of features disclosed in this specification. It will be also be appreciated that, throughout this specification, language in the general form of “X for Y” (where Y is some action, activity or step and X is some means for carrying out that action, activity or step) encompasses means X adapted or arranged specifically, but not exclusively, to do Y.

Each feature disclosed in this specification may be replaced by alternative features serving the same, equivalent or similar purpose, unless expressly stated otherwise. Thus, unless expressly stated otherwise, each feature disclosed is one example only of a generic series of equivalent or similar features.

The reader's attention is directed to all papers and documents which are filed concurrently with or previous to this specification in connection with this application and which are open to public inspection with this specification, and the contents of all such papers and documents are incorporated herein by reference.

1. A bone cement pressuriser comprising:
a pressuriser head having a first side coupled to or configured to couple to an end of a pressuriser handle and a second side configured to transfer pressure to bone cement in a bone cavity;
wherein the second side of the pressuriser head is defined by a first, convex curved surface and a second surface forming a ring surrounding the first surface and extending radially beyond the first surface; and
wherein the first surface is configured to pressurise bone cement within a bone cavity and the second surface is configured to both pressurise bone cement within the bone cavity and to seal about the periphery of the bone cavity.

2. A bone cement pressuriser according to claim 1, wherein the pressuriser head comprises a filled volume between the first and second sides.

3. A bone cement pressuriser according to claim 1, wherein the pressuriser head is shaped to pressurise bone cement in an acetabulum;
wherein the first surface is shaped to excavate bone cement within the acetabulum under the application of pressure; or
wherein the second surface is shaped to seal the periphery of the acetabulum to minimise the egress of bone cement.

4. A bone cement pressuriser according to claim 1, wherein the second surface flares outwards from the first surface; or
wherein the second surface extends directly from the first surface to define a continuous surface.

5. A bone cement pressuriser according to claim 1, wherein the first and second surfaces are uninterrupted.

6. A bone cement pressuriser according to claim 1, wherein the second surface is also a convex curved surface, the first surface being more tightly curved than the second surface.

7. A bone cement pressuriser according to claim 1, wherein the first surface is domed or defined by a spheroidal cap.

8. A bone cement pressuriser according to claim 1, wherein the first and second surfaces form a double-domed surface.

9. A bone cement pressuriser according to claim 1, wherein the first or second surface is rotationally symmetrical.

10. A bone cement pressuriser according to claim 1, wherein the first surface is defined by a spheroidal cap having a first focus defining a first polar axis; and
wherein the second surface is defined by a spheroidal segment having a second focus located on the first polar axis.

11. A bone cement pressuriser according to claim 10, wherein the first and second centres are spaced apart along the first polar axis;
wherein the second centre is further from the second side of the pressuriser head than the first centre;
wherein the gap between the first and second centres is greater than or equal to about 5 mm; or
wherein the pressuriser handle extends at least partially along the first polar axis.

12. A bone cement pressuriser according to claim 10, wherein the spheroidal cap is a spherical cap having a first radius between about 15 mm and about 40 mm.

13. A bone cement pressuriser according to claim 12, wherein the first radius is approximately 26 mm.

14. A bone cement pressuriser according to claim 10, wherein the spheroidal segment is a spherical segment having a second radius greater than or equal to about 20 mm.

15. A bone cement pressuriser according to claim 10, wherein the maximum width of the second surface along an axis normal to the first polar axis is between about 37 mm and about 150 mm.

16. A bone cement pressuriser according to claim 1, wherein the pressuriser head further comprises a flange surrounding the second surface or an asymmetric protrusion extending from the second surface.

17. A bone cement pressuriser according to claim 1, further comprising a pressuriser handle configured to couple to the first side of the pressuriser head.

18. A bone cement pressuriser according to claim 1, wherein the pressuriser head is elastically deformable.

19. A bone cement pressuriser according to claim 1, wherein the pressuriser head is formed from a viscoelastic material with a Shore A hardness between about 0 and 90.

20. A bone cement pressuriser according to claim 19, wherein the pressuriser head is formed from silicone.

21. A method of pressurising bone cement, the method comprising:
preparing a bone cavity for receiving bone cement;
inserting bone cement into the bone cavity; and
pressurising the bone cement in the bone cavity using a bone cement pressuriser according to claim 1 by bearing the second side of the pressuriser head against exposed bone cement in the bone cavity and applying pressure to the pressuriser head through a coupled pressuriser handle;
wherein the first surface is configured to pressurise bone cement within a bone cavity and the second surface is configured to both pressurise bone cement within the bone cavity and to seal about the periphery of the bone cavity.
